(19) United States
(12) Patent Application Publication
(10) Pub. No.: US 2020/0188450 A1
(43) Pub. Date: Jun. 18, 2020

(54) FECAL MICROBIOTA FOR TREATING PATIENTS UNDERGOING A HEMATOPOIETIC STEM CELL TRANSPLANT

(71) Applicants: SORBONNE UNIVERSITE, Paris (FR); CENTRE NATIONAL DE LA RECHERCHE SCIENTIFIQUE, Paris (FR); INSERM (INSTITUT NATIONAL DE LA SANTE ET DA LA RECHERCHE MEDICALE), Paris (FR); ASSISTANCE PUBLIQUE - HOPITAUX DE PARIS, Paris (FR)

(72) Inventors: Mohamad MOHTY, Paris (FR); Harry SOKOL, Paris (FR)

(21) Appl. No.: 16/480,863

(22) PCT Filed: Jan. 26, 2018
(86) PCT No.: PCT/EP2018/051941

§ 371 Date: Jul. 25, 2019

(30) Foreign Application Priority Data
Jan. 26, 2017 (FR) 1750629

(51) Int. Cl. A61K 35/74

(52) U.S. Cl. CPC A61K 35/74

(57) ABSTRACT

The invention relates to a fecal microbiota sample for use in the prevention and/or treatment of infectious or non-infectious complications resulting from allogeneic hematopoietic stem cell (HSC) transplantation, or for the treatment of cancer in a recipient patient, said fecal microbiota sample and said hematopoietic stem cells originating from the same donor subject, or said fecal microbiota sample being administered prior to HSC transplantation.

The invention relates to the treatment of patients undergoing a hematopoietic stem cell (HSC) transplant.

### TECHNOLOGICAL BACKGROUND

Hematopoietic stem cells are cells at the origin of all blood cell lines. By differentiation, these cells are capable of giving rise to any blood cell (red corpuscles, white corpuscles, platelets) and are also capable of self-renewal.

Hematopoietic stem cell transplant is a major therapeutic method in the treatment of certain blood disorders and certain cancers. In fact, it allows the intensity of treatment by chemotherapy and/or radiotherapy to be increased to massive doses, resulting in treatment of the disorder, or recovery, with an improvement in patient survival. It also allows a new immune system to be introduced in the patient (the case of allograft) which can contribute to the control of the cancer disease.

A transplant carried out after these treatments which involve significant hematological toxicity allows reconstruction of the bone marrow and the return to normal production of blood cells. Hematopoietic stem cell transplant is used in the treatment of several types of pathologies: malignant, such as acute leukemias, myelomas, lymphomas or certain solid tumors (breast cancer, testicular cancer, ovarian cancer, neuroblastoma, etc.) and also non-malignant, such as congenital or acquired deficiencies (immune deficiency, aplasia, metabolic deficiencies and errors of metabolism, etc.).

Two types of transplant can be performed:
autograft, where the patient receives their own stem cells taken from the bone marrow, or from the peripheral blood several weeks, months or years earlier, and stored frozen.
allograft, (or “allogenic graft”) which requires a related donor, compatible or not, or unrelated, compatible or not, or umbilical cord blood cells.

The transplant is carried out in several steps. Several days before undergoing the transplant, the patient is typically subjected to a “conditioning” that is myeloablative or not myeloablative. This is often more or less severe chemotherapy and optionally total body irradiation in order to destroy the bone marrow of the recipient and allow the transplant to take. Generally, the aim is to destroy all the cells present in the bone marrow of the recipient in order to allow the hematopoietic stem cells of the donor to develop therein and thus to substitute for the destroyed bone marrow of the recipient.

The transplant of hematopoietic cells then takes place by intravenous transfusion. The transplant is followed by a period of aplasia during which the patient no longer has immune defenses.

In the case of allografts, the “conditioning” period preceding the allogenic graft makes it possible to create sufficient immunosuppression to prevent rejection of the graft, and if necessary, to obtain an antineoplastic effect on the malignant cells. However, this beneficial effect is counterbalanced by the high risks of graft-versus-host (GVH) disease linked to the immunological reactions between the donor and the recipient. Furthermore, the “conditioning” exposes the patient to risks of bacterial, viral or fungal infections, requiring prophylactic or therapeutic anti-infectious treatments.

To date, no treatment for these serious complications is fully satisfactory.

There is still a need for a preventive or therapeutic approach for such complications.

### SUMMARY OF THE INVENTION

In response to this therapeutic need, the inventors propose to combine the HSC transplant with fecal microbiota transplantation. The inventors more particularly identified the benefit where the fecal microbiota donor is the same as the HSC donor. They also identified the benefit of a fecal microbiota transplantation before the HSC transplant, whether in the case of an isologous situation (where the fecal microbiota donor and the HSC donor are identical) or in the case of a situation where the fecal microbiota donor and the HSC donor are different.

An aspect of the invention thus relates to the use of a sample of fecal microbiota in the prevention and/or the treatment of infectious or non-infectious complications resulting from an allogenic HSC transplant in a recipient patient, said sample of fecal microbiota and said HSC originating from one and the same donor subject.

The fecal microbiota transplantation can be carried out before or after the HSC allograft.

Another aspect of the invention relates to the use of a sample of fecal microbiota in the prevention and/or the treatment of infectious or non-infectious complications resulting from an allogenic HSC transplant in a recipient patient, said sample of fecal microbiota being administered before the HSC transplant.

Advantageously, the fecal microbiota makes it possible to prevent the occurrence, or reduce the risk of occurrence, of a graft-versus-host (GVH) disease following the allogenic HSC transplant.

The fecal microbiota also makes it possible to reduce the risk of an infectious complication, which may even include septicemia, which would be due to colonization by pathogenic bacteria, in particular bacteria that are multiresistant to antibiotics, having infected the recipient patient before the allogenic HSC transplant, and even before the myeloablative or non-myeloablative conditioning of the patient has started. Such an infection is typically a contraindication for the allogenic HSC transplant. Fecal microbiota transplantation according to the invention resolves this contraindication, the patient becoming able to receive conditioning and an allogenic HSC transplant.

The recipient patient may in particular suffer from a cancer, in particular a malignant hemopathy, but also a non-malignant disorder.

Another aspect of the invention relates to the use of a sample of fecal microbiota in the treatment of a cancer in a recipient patient suffering from a cancer, in particular a malignant hemopathy, and undergoing or having undergone an allogenic HSC transplant, said sample of fecal microbiota and said HSC preferably originating from one and the same donor subject.

Here again, the fecal microbiota transplantation can be carried out before or after the HSC transplant.

### DETAILED DESCRIPTION OF THE INVENTION

The Recipient Patient

The patient envisaged is a human, regardless of age and sex, who has undergone, or will undergo, a hematopoietic stem cell transplant. The patient suffers from any disorder that can be treated by an HSC transplant. These are in particular cancers, genetic disorders, disorders affecting the hematopoietic system and/or the immune system. There may be mentioned in particular: solid tumors, malignant hemopathies, including chronic myeloid leukemia, acute myeloid leukemia, acute lymphoblastic leukemia, myeloproliferative syndromes, myelodysplasic syndromes, NHL (non-Hodgkin malignant lymphomas), Hodgkin's disease, multiple myeloma, idiopathic medullary aplasia, paroxysmal nocturnal hemoglobinurea, hemoglobinopathies, congenital immune deficiencies (SCID, Kostmann, Wiskott-Aldrich) and Fanconi anemia.

The patient undergoes a conditioning (or preparation) before the hematopoietic stem cell transplant, which may be a myeloablative or non-myeloablative treatment.

A review of this type of treatment is presented in Petersen et al, 2007 (Alloreactivity of therapeutic principle in the treatment of hematologic malignancies. Danish Medical Bulletin, May 2007, 54: 112-39). An example of myeloablative treatment can be as follows: cyclophosphamide (120 mg/kg) combined with busulfan (16 mg/kg) or with total body irradiation of the patient with an absorbed radiation dose of 12 Gy.

In the case of non-myeloablative treatment, the patient can have total body irradiation with a radiation dose less than 12 Gy or receive chemotherapy based on cyclophosphamide and/or fludarabine. For example, it is possible to provide a non-myeloablative lymphopenic treatment, which comprises an intravenous injection of cyclophosphamide 50 mg/kg once on D-6 and D-5, and injection of fludarabine 25 mg/m² from D-6 to D-2 (DO being the day of the hematopoietic stem cell transplant). Cf Sandmaier et al, Semin Oncol. 2000 April: 27 (2 Suppl 5): 78-8.

Sequential conditioning with Thiotepa is also possible, as described in Duléry et al, Biol Blood Marrow Transplant. 2018 Jan. 11. p ii1083-8791(18)).

The hematopoietic stem cells used can originate from any source, for example from peripheral blood or bone marrow, from a donor subject.

The Fecal Microbiota

The invention implements a fecal microbiota transplantation which according to certain embodiments can advantageously originate from the same donor subject as the HSC donor subject.

By “fecal microbiota” is meant the microbial, in particular bacterial, flora present in the stools of a healthy individual. The human intestinal microbiota is the body of microorganisms (bacteria, yeasts and fungi) that are found in the human gastrointestinal system (stomach, intestine and colon). Microbial diversity is currently estimated at around 10 bacterial species composing the dominant intestinal microbiota of an adult individual, with an abundance of 1014 bacteria, representing a bacterial metagenome of 200,000 to 800,000 genes in each individual, i.e. 10 to 50 times the number of genes of the human genome. Although sterile in utero, the intestine is colonized from the first days of life, evolving towards a unique individual microbiota. Each person has bacteria that are relatively close in terms of species, but the exact composition of their microbiota (species, proportions) is largely (over ¾ of the species) specific to the host.

By “transplantation of fecal microbiota” or “transplantation of a sample of fecal microbiota” is meant the administration of fecal microbial, in particular bacterial, flora from a healthy individual into a patient.

In the context of the present invention, the “recipient patient” of the hematopoietic stem cell transplant is different from the “donor subject” of the hematopoietic stem cells and from the “donor subject” of the sample of fecal microbiota, it being understood that in certain particular embodiments, the donor subject of the hematopoietic stem cells is also the donor subject of the sample of fecal microbiota. The human donor subject of hematopoietic stem cells preferably has good histocompatibility with the recipient patient, namely that the donor has an HLA typing as close as possible to the recipient. Thus, it is preferable for at least 5 to 10 HLA antigens to be compatible.

Preferably, the donor subject of the fecal microbiota is a healthy subject. By “healthy subject” is meant a subject not suffering from an imbalance of the intestinal microbiota or a pathology diagnosed/recognized by the medical profession. Also preferably, the donor subject of fecal microbiota is a close relative of the patient, preferably a family member, living in the same environment.

The sample of fecal microbiota typically comprises bacteria from the Blautia, Ruminococcus, Eubacterium, Holdemania and Clostridium genus, in particular bacteria from the species Ruminococcus obeum, Clostridium hathewayi, Eubacterium desmolans, Dorea longicatena, Ruminococcus lactaris (Blautia producta), Eubacterium contortum, Ruminococcus faecis, Holdemania filiformis, Clostridium sordelli.

The sample of fecal microbiota can be obtained and stored by any means known to a person skilled in the art, while advantageously taking care to preserve the viability of the bacteria constituting the fecal microbiota, in particular the anaerobic bacteria.

This step is carried out preferably by taking a stool sample from the donor subject. In fact, the stool sample contains fecal microbiota from the donor subject. Thus, the method comprises a step of taking at least one stool sample, comprising the fecal microbiota, from the donor subject. Preferably, the stool sample has a weight of at least 10 g.

The sample of fecal microbiota can be frozen, for use in a thawed condition, or be prepared from fresh stools. A mixture of fresh stools and frozen sample is also possible.

According to a particular embodiment, a sample is used in the invention that can be obtained by thawing from i) mixing a stool sample from the donor subject with a saline aqueous solution comprising a cryoprotectant agent and/or a bulking agent, optionally followed by ii) filtration, before iii) freezing for storage, the steps of mixing and freezing preferably being carried out under anaerobic conditions.

Such a method for the preparation of a sample of fecal microbiota is for example described in patent application WO2016/170285.

It can in particular comprise the following steps:

a) taking at least one sample of fecal microbiota from the donor subject,

b) placing said sample obtained in a) in a collection device, preferably sealed against oxygen, preferably within a time limit of less than 5 minutes after taking the sample,

c) mixing the sample obtained in b) with at least one saline aqueous solution comprising at least one cryoprotectant and/or a bulking agent,

d) optionally, filtering the mixture obtained in c), in particular by a filter comprising pores of diameter less than or equal to 0.7 mm, preferably less than or equal to 0.5 mm and

e) storing the mixture obtained in c) or d) by freezing at a temperature typically comprised between −15° C. and −100° C., preferably between −60° C. and −90° C.,

steps b) to e) preferably being carried out anaerobically.

Once the sample (obtained in a)) has been placed in a collection device, preferably sealed against oxygen, it can optionally be incubated at a temperature comprised between 33° C. and 40° C. for a maximum period of 75 hours. Preferably, this step of incubation is carried out at a temperature comprised between 35° C. and 38° C. for a period comprised between 24 hours and 73 hours. Ideally, this step takes place at a temperature of approximately 37° C. for 72 hours. Alternatively, the sample can optionally be incubated at a temperature comprised between 2° C. and 10° C. for a maximum period of 75 hours. Preferably, this step of incubation is carried out at a temperature comprised between 4° C. and 8° C. for a period comprised between 24 hours and 72 hours.

Next comes step c): this step comprises mixing the sample obtained in b) with at least one saline aqueous solution comprising at least one cryoprotectant and/or a bulking agent.

Typically, the saline aqueous solution comprises water and physiologically acceptable salts. Typically, the salts are calcium, sodium, potassium or magnesium salts, with chloride, gluconate, acetate or hydrogen carbonate ions.

The saline aqueous solution can also optionally comprise at least one antioxidant. The antioxidant is selected in particular from ascorbic acid and salts thereof (ascorbate), tocopherols (in particular—tocopherol), cysteine and salt forms thereof (in particular hydrochloride) and mixtures thereof.

Preferably, the saline aqueous solution comprises:
at least one salt selected from sodium chloride, calcium chloride, magnesium chloride, potassium chloride, sodium gluconate and sodium acetate, and
optionally at least one antioxidant, preferably selected from sodium L-ascorbate, the tocopherols, L-cysteine hydrochloride monohydrate and mixtures thereof.

Typically, the salt is present in the saline aqueous solution in a concentration comprised between 5 and 20 g/L, preferably between 7 and 10 g/L.

Typically, the antioxidant is present in the saline aqueous solution in a quantity comprised between 0.3 and 1% by weight with respect to the total volume of solution, preferably in a quantity comprised between 0.4 and 0.6% by weight with respect to the total volume of solution. Preferably, when the antioxidant is a mixture of sodium L-ascorbate and L-cysteine hydrochloride monohydrate, the sodium L-ascorbate is present in a quantity comprised between 0.4 and 0.6% by weight with respect to the total volume of solution, and the L-cysteine hydrochloride monohydrate is present in a quantity comprised between 0.01 and 0.1% by weight with respect to the total volume of solution.

Preferably, the saline aqueous solution also comprises at least one cryoprotectant. A cryoprotectant is a substance used in order to protect the sample from damage caused by freezing, in particular due to the formation of ice crystals.

Preferably, the cryoprotectant is selected from the polyols, di- to pentasaccharides (i.e. the disaccharides, trisaccharides, quadrisaccharides and pentasaccharides), DMSO and mixtures thereof. Preferably, the cryoprotectant is selected from the polyols, tri- and disaccharides, DMSO and mixtures thereof. More preferentially, the cryoprotectant present in the saline aqueous solution is a disaccharide or a trisaccharide.

Among the usable polyols, there are in particular glycerol, mannitol, sorbitol and also propylene glycol or ethylene glycol. Among the usable di- to pentasaccharides, there may be mentioned the dimers, trimers, quadrimers and pentamers having identical or different units, said units being selected from glucose, fructose, galactose, fucose and N-acetylneuraminic acid. Among the usable disaccharides, there are in particular trehalose or one of the analogues thereof, or saccharose. Finally, DMSO, or dimethylsulfoxide, is a standard cryoprotectant.

These cryoprotectants can be used alone or in a mixture.

Typically, the total quantity of cryoprotectant present in the saline aqueous solution is comprised between 3 and 30% by weight with respect to the total volume of solution, preferably between 4% and 20% by weight with respect to the total volume of solution.

Preferably, the cryoprotectant is selected from glycerol, mannitol, sorbitol, DMSO, propylene glycol, ethylene glycol, trehalose and analogues thereof, saccharose, galactose-lactose and mixtures thereof. More preferentially, the cryoprotectant is galactose-lactose or trehalose. Preferably, the saline aqueous solution comprises at least one bulking agent.

The bulking agent is preferably selected from the partial hydrolysates of starch or of fecula. The partial hydrolysates of starch, in particular of wheat or maize, as well as the partial hydrolysates of fecula, for example potato, comprise a high quantity of maltodextrins. The maltodextrins are the result of partial hydrolysis of starch or of fecula, and are constituted by different sugars (glucose, maltose, maltotriose, oligo- and polysaccharides), the proportions of which vary as a function of the degree of hydrolysis.

Preferably, the bulking agent present in the saline aqueous solution is a mixture of maltodextrins, in which the quantity of maltodextrins is comprised between 4 and 20% by weight with respect to the total volume of solution.

Preferably, the saline aqueous solution comprises both:
at least one cryoprotectant as described above, i.e. selected from the polyols, di- to pentasaccharides (i.e. the disaccharides, trisaccharides, quadrisaccharides and pentasaccharides), DMSO and mixtures thereof, and
at least one bulking agent as described above, i.e. selected from the partial hydrolysates of starch or of fecula, preferably the bulking agent is constituted by maltodextrins.

Preferably, in this case, the quantity of cryoprotectant is comprised between 3 and 30% by weight with respect to the total volume of solution, preferably between 4% and 20% by weight with respect to the total volume of solution; and the quantity of bulking agent, preferably of maltodextrins, is comprised between 4 and 20% by weight with respect to the total volume of solution.

Step c) of mixing the sample obtained in b) with at least one saline aqueous solution comprising at least one cryoprotectant can in particular be carried out by mixing, in order to obtain a homogenous mixture.

Preferably, the sample obtained in b) is mixed with said saline aqueous solution in a respective weight/volume ratio comprised between 0.5 by weight:10 by volume and 2 by weight:2 by volume. A sample:solution weight/volume ratio equal to 0.5 by weight:10 by volume means that the sample is mixed at 0.5 by weight (for example 0.5 g) to 10 by volume of solution (for example 10 ml). Preferably, the sample:solution weight/volume ratio is equal to 1 by weight of sample to 4 by volume of solution (1 by weight:4 by volume).

Once this step has been carried out, an optional step d) can be performed. Step d) comprises filtering the mixture obtained in c), in particular by a filter comprising pores of diameter less than or equal to 0.7 mm, preferably less than or equal to 0.5 mm. Such a filtration allows the coarse particles to be retained and the bacteria of interest (constituting the fecal microbiota) to be retained in the filtrate.

Then, following step c) or d), when the latter takes place, the mixture obtained is stored by freezing at a temperature comprised between −15° C. and −100° C.: this is step e). Preferably, the temperature of freezing (and therefore of storage) is comprised between −60° C. and −90° C.; more preferentially it is approximately −80° C. or approximately −65° C.

In order to be frozen, following step c) or d) and before step e), the mixture can be aliquoted beforehand, in order to ensure specimens having a constant volume. For example, the aliquoting is carried out in order to obtain specimens having a volume equal to 50 ml, 100 ml, 150 ml, or 200 ml. Preferably, the aliquoting is carried out in order to obtain specimens having a volume equal to 100 ml.

This step of freezing and storage makes it possible to store the treated samples for a period of at least 2 months. The samples stored in this way are also good-quality, even after thawing. Preferably, the method comprises a step 0 of thawing the frozen sample obtained in e), anaerobically, up to ambient temperature. This thawing step f) can be carried out by placing the frozen sample in a water bath at a temperature comprised between 35° C. and 40° C., for example 37° C., for a period of several minutes (typically from 2 to 10 minutes). The thawing step f) can also be carried out by placing the frozen sample at a temperature comprised between 2° C. and 10° C., for example between 4° C. and 8° C., for a period of 10 to 20 hours.

The sample thus thawed, at ambient temperature, can then be administered to the recipient patient.

In a preferred embodiment, the stools are handled at a microbiological security station in the 6 hours after emission. The stools from the donor are weighed and mixed with a saline cryopreservation solution (glycerol+NaCl 0.9%, 10/90 V/V) by using a weigh scales, a blender, sterile containers and sterile medical equipment (syringes, filters, etc.). The homogenization is carried out in 500 ml for 50-100 g of stools. The suspension obtained is filtered by using a funnel with sterile dressing gauze and sterile containers in order to remove all solid residue before freezing at −80° C.

On the day before the fecal transplantation, the frozen solution is placed in the refrigerator (between 4 and 8° C.) in order to thaw overnight. On the day of the fecal transplantation, the solution is placed in an enema bag. The material is ready for use.

In another embodiment, it is possible to use fresh stools, i.e. not frozen.

In a preferred embodiment, the stools are handled at a microbiological security station in the 6 hours after emission. The stools from the donor are weighed and mixed with a sterile saline solution (NaCl 0.9%, 10/90 V/V) by using a weighing scale, a blender, sterile containers and sterile medical equipment (syringes, filters, etc.). The homogenization is carried out in 500 ml for 50-100 g of stools. The suspension obtained is filtered by using a funnel with sterile dressing gauze and sterile containers in order to remove all solid residue. The solution is then placed in an enema bag. The material is ready for use.

Therapeutic Indications

A method for the prevention and/or the treatment of infectious or non-infectious complications resulting from an allogenic hematopoietic stem cell transplant (HSC) in a recipient patient is described herein, comprising the administration to said recipient patient of a sample of fecal microbiota, said sample of fecal microbiota and said hematopoietic stem cells preferably originating from one and the same donor subject. By infectious or non-infectious complications resulting from an allogenic HSC transplant” is meant in particular the complications due to the conditioning of the patient for the allogenic HSC transplant, conditioning that reduces the immune defenses of the patient, as well as the complications due to the allogenic transplant itself, i.e. in particular graft-versus-host (GVH) disease in its acute and/or chronic forms.

A method is also described for the treatment by hematopoietic stem cell transplant (HSC) of a patient having a need for such a treatment, in which a suitable quantity of sample of fecal microbiota is administered to said patient, before and/or after the HSC transplant, said sample of fecal microbiota preferably originating from one and the same donor individual as the HSC.

The term “treatment” includes a therapeutic treatment resulting in recovery or a palliative treatment, improving, eliminating, reducing and/or stabilizing the symptoms of a disorder or pain thereby caused.

The term “prevention” corresponds to a prophylactic or preventive treatment, which corresponds equally well to a treatment resulting in the prevention of a disorder as to a treatment reducing and/or retarding the incidence of a disorder or the risk of development thereof.

In the context of the present invention, the transplantation of a fecal sample reduces the risk for the recipient patient of developing one or more infectious or non-infectious complications following the HSC transplant, and/or improves or cures one or more of the complications arising after the HSC transplant.

The fact that the sample of fecal microbiota originates from the same donor as the HSC allows better tolerance of the fecal microbiota transplantation (FMT) by the patient, in the case where the FMT takes place after the HST transplant. In fact, during the prior HSC transplant, the immune cells of the donor are transferred to the recipient patient. The immune reactions during the FMT are therefore reduced by the fact of the presence of immune cells from this same donor in the body of the recipient patient.

An FMT carried out before the HSC transplant makes it possible to reduce, or to eliminate, any infections by resistant bacteria, for example Citrobacter freundii or Pseudomonas aeruginosa, that the candidate patient may be carrying, before the HSC transplant and the prior conditioning therefor.

It also allows a correction of the intestinal microbiota of the candidate patient for the HSC transplant, and therefore makes it possible to reduce the incidence of the complications that may occur after the HSC transplant.

The invention also makes it possible to reduce courses of antibiotics with respect to infections. The infectious complications are in particular an infection by Clostridium or by enterococci, in particular an infection by vancomycin-resistant enterococci (VRE) or any other germ resistant to the classical antibiotics.

The Clostridium difficile infections, in particular recurrent infections, are particularly targeted. Among the other opportunistic species that can cause infectious complications there may be mentioned E. fecalis, P. mirabilis, or E. coli.

The most well-known non-infectious complication is the graft-versus-host disease (GVH). Acute GVH generally occurs within the 6 months following the HSC transplant and may have a frequency comprised between 10 and 80% of cases, depending on the genetic disparity between the donor and recipient. Chronic GVH is generally so termed when it occurs over 100 days after the transplant or also as a function of certain clinical characteristics of this GVH. Other complications include enteropathies and other digestive complications post-transplant.

According to another aspect, a method is described for treating a cancer in a recipient patient suffering from a cancer and undergoing or having undergone an allogenic hematopoietic stem cell transplant, the method comprising the administration of a sample of fecal microbiota to said patient, said sample of fecal microbiota and said hematopoietic stem cells preferably originating from one and the same donor subject.

The term “treat” or “treatment” includes slowing the progression of cancer, complete remission, or reduction in the risk of relapse.

This can in particular involve a solid tumor or a malignant hemopathy, as mentioned above.

In fact, the fecal microbiota transplantation and the HSC transplant from the same donor can offer a synergistic effect with respect to the elimination of the residual cancer cells, and therefore persistent remission of the cancer. Indeed, the HSC contain immunocompetent cells (Natural Killer cells, lymphocytes B and T in particular), which have already been in contact with the environment of the donor. These HSC therefore contain lymphocytes and other specific immune effectors pre-activated by the antigens of the commensal bacteria (of the intestinal flora) of the donor. Without being bound to a particular mechanism of action, the inventors hypothesize that these pre-activated immune effectors transplanted into the recipient are re-stimulated by the transplanted bacteria.

A combination product (“kit of parts”) is also described here comprising on the one hand human hematopoietic stem cells (HSC) and on the other hand a sample of fecal microbiota, the HSC and the sample of fecal microbiota preferably originating from one and the same donor individual, for combined use simultaneously or staggered over time in a treatment by HSC transplant as described above.

A kit can also be supplied, comprising on the one hand human hematopoietic stem cells (HSC) in a suitable packaging and on the other hand a sample of fecal microbiota in a suitable packaging, the HSC and the sample of fecal microbiota preferably originating from one and the same donor individual. The methods of taking and packaging the HSC on the one hand and the samples of fecal microbiota on the other hand are well known to a person skilled in the art, and in particular described above. By “kit” is meant according to the invention that both elements of which it is constituted are associated for a combined use, and that they can be stored together or separately for use thereof in one and the same patient.

Protocols

The sample of fecal microbiota can be administered (the term “transplanted” is also used) in the alimentary tract of the recipient patient before or after said HSC transplant.

The patient can receive a single administration of the sample of fecal microbiota, or repeated administrations, for example from 4 to 8 days or from 2 to 8 weeks apart.

In a particular embodiment, the sample of fecal microbiota is transplanted in the alimentary tract of the recipient patient from 1 week to 2 years, preferably from 1 week to 1 year, more preferably from 1 week to 6 months, and even more preferably from 1 week to 3 months, after said HSC transplant, optionally repeatedly.

In another particular embodiment, the sample of fecal microbiota is transplanted in the alimentary tract of the recipient patient from 1 week to 12 months, preferably 1 week to 8 months, preferably 1 week to 6 months, more preferably from 1 week to 4 months, preferably 1 week to 3 months, or from 1 to 8 weeks, preferably from 1 to 6 weeks, before said HSC transplant, optionally repeatedly.

Generally, the quantities administered are such that they allow the colonization of the digestive tract by the bacteria and other microorganisms of the implanted fecal microbiota.

The quantities administered are typically comprised between 100 ml and 1 L, preferably approximately 500 ml, of saline solution prepared from a fecal sample, as described above. Preferably, the sample of fecal microbiota comprises 10¹⁰ to 10¹³ bacteria/g of sample.

The transplantation of the sample of fecal microbiota can be carried out by an administration via the rectal route, via the oral route (for example in freeze-dried form, or in the form of capsules) or by any method allowing the transfer of the microbiota into the intestinal tract. For example, enema bags can be used for this purpose. The transplantation of the sample of fecal microbiota can also be carried out by means of a colonoscope, a device normally used for examining the colon.

The following examples illustrate the invention without limiting the scope thereof.

### Example 1: Transplantation of Isologous Fecal Microbiota Post-HSC Transplant in a Female Patient Suffering from Acute Myeloid Leukemia

Protocol:

The patient is a 16-year-old girl suffering from acute myeloid leukemia, having relapsed after a first HSC allograft.

This patient received a second HSC transplant originating from her mother, after a conditioning of reduced intensity including a combination of chemotherapy drugs. The patient received 5 mg/kg of Thiotepa six days before the transplant, then between five and two days before the transplant, fludarabine 40 mg/m²/day and, between 5 and 4 days before the transplant, busulfan at 3.2 mg/kg/day. Finally, between three and five days after the transplant, the patient received 50 mg/kg/day of cyclophosphamide.

The patient did not develop an acute graft-versus-host reaction immediately after the transplant and it was possible to stop her immunosuppressant treatment based on cyclosporin-A and mycophenolate mofetil 83 days after the transplant.

However, multiple bacterial complications (sepsis, multiresistant bacteria) were observed during the first months after the graft, requiring the use of broad-spectrum antibiotics. One month after the transplant, the patient developed a first episode of Clostridium difficile infection which was successfully treated with metronidazole. Two and a half months after the transplant, the presence of vancomycin-resistant enterococci (VRE) was detected by rectal swabbing. Due to the recurrence of a Clostridium difficile infection resistant to treatment by metronidazole and fidaxomicin, a first fecal microbiota transplantation (originating from the mother of the patient, also HSC donor) was then carried out, 3 months after the HSC transplant.

The stools from the mother had been mixed beforehand with a saline cryopreservation solution (glycerol+NaCl 0.9%, 10/90 V/V). The homogenization was carried out in 500 ml for 50-100 g of stools. The suspension obtained was filtered by using a funnel with sterile dressing gauze and sterile containers in order to remove all solid residue. The solution was then placed in an enema bag, ready for use.

Results:

The gastrointestinal symptoms reduced markedly, and examinations showed recovery from the Clostridium difficile infection and eradication of the vancomycin-resistant enterococci.

Six months after the HSC transplant, the patient presented with pancytopenia associated with sepsis due to a pathogenic bacterium, Klebsiella pneumoniae, producing broad-spectrum beta-lactamases, and VRE carrier status. In addition, the patient presented with abdominal pain and significant diarrhea, without demonstrating a Clostridium infection or gastrointestinal graft-versus-host reaction.

A second fecal microbiota transplantation (FMT) from the same donor (the mother) was practiced on the patient in order to treat the VRE colonization. After the FMT, the clinical signs improved and the VRE was not longer detectable.

No multiresistant bacteria (in particular Clostridium or VRE) were detected by rectal swabbing 12 months after the HSC transplant.

3 years after the HSC transplant, the patient is in full remission from the acute leukemia.

### Example 2: Transplantation of Isologous Fecal Microbiota Post-HSC Transplant in a Female Patient Suffering from Acute Lymphoblastic Leukemia

The patient was a 19-year-old girl suffering from acute lymphoblastic leukemia in relapse after several courses of chemotherapy, when she received an HSC transplant from her mother (haploidentical), with sequential conditioning with Thiotepa, as subsequently described in Duléry et al., supra.

Following this transplant, she developed acute GVH (treated), then chronic GVH.

Four months after the HSC transplant, she was detected positive for vancomycin-resistant enterococci (VRE).

Six and eight months after HSC, she received a fecal microbiota transplantation, also from her mother, the sample being prepared as described in Example 1.

No multiresistant bacteria were subsequently detected by rectal swabbing.

The signs of GVH were in the process of improvement when the patient's leukemia relapsed, two years after the HSC transplant.

### Example 3: Transplantation of Isologous Fecal Microbiota Pre-HSC Transplant in a Male Patient Suffering from Acute Dendritic Cell Leukemia

The 47-year-old patient diagnosed with acute dendritic cell leukemia underwent chemotherapy (methotrexate, idarubicin et asparaginase combination in the induction phase, then two consolidation phases). This patient tested positive to Citrobacter freundii, which can be a contraindication for an HSC transplant.

He received a fecal microbiota transplantation originating from his HLA-identical sister, which resolved the Citrobacter freundii colonization, making the patient able to receive an HSC transplant. The HSC transplant (also from his sister) was carried out 9 days later, after a conditioning including Thiotepa, Busulfan and Fludarabine.

The stools from the sister were prepared for the fecal microbiota transplantation as explained in Example 1.

The patient did not develop any acute graft-versus-host reaction immediately after the HSC transplant and it was possible to stop his immunosuppressant treatment based on cyclosporin 4 months after the allograft.

The bacterial infection complications (including a vancomycin-resistant staphylococcus infection (VRSA)) were limited and controlled.

One year after the transplant, the patient is in remission.

### Example 4: Transplantation of Non-Isologous Fecal Microbiota Pre-HSC Transplant in a Male Patient Suffering from Acute Myeloid Leukemia

This 64-year-old patient suffering from acute myeloid leukemia was first treated with two chemotherapy lines.

During the induction treatments of the chemotherapy, this patient developed a severe sepsis from Pseudomonas aeruginosa.

A fecal microbiota transplantation was carried out from stools from his daughter, prepared according to the protocol of Example 1, which allowed the negativation of the multiresistant germ.

An allograft of peripheral stem cells from an HLA-identical donor was carried out 41 days later, in the absence of major infectious episodes, and after conditioning with Busulfan and Fludarabine.

No acute digestive GVH was observed following the transplant.

The patient is in remission (2 years after the HSC transplant).

### Example 5: Transplantation of Non-Isologous Fecal Microbiota Pre-HSC Transplant in a Male Patient Suffering from Acute Myeloid Leukemia

This 42-year-old patient was suffering from an acute myeloid leukemia that was resistant to the first chemotherapy line but responded to the second chemotherapy line.

This patient showed a multiresistant Pseudomonas aeruginosa colonization, without associated systemic infectious complications, however from the perspective of an HSC allograft and the consequent immunosuppressive state, a decision was taken to carry out a fecal microbiota transplantation from his sister. This FMT was carried out according to the protocol of Example 1. 46 days later, and following a sequential conditioning with Thiotepa, as described in Duléry et al, supra, this patient was able to receive an HSC transplant from his haploidentical brother. No particular complication (neither GVH nor infection) was observed after the transplant. However, this patient had a relapse of leukemia and died 11 months after the HSC transplant.

### Example 6: Transplantation of Non-Isologous Fecal Microbiota Pre-HSC Transplant in a Female Patient Suffering from Acute Myeloid Leukemia

This 45-year-old patient was diagnosed at high-risk with acute myeloid leukemia and treated with two lines of chemotherapy in Mauritius.

She arrived in France for an HLA-identical HSC allograft from her sister.

During the period of hospitalization for the pre-transplant assessment and to resolve multiple cardiopulmonary comorbidity problems, a resistant Citrobacter freundii was detected on rectal swabbing. It should be noted that at the same time the patient was colonized by a resistant Klebsiella pneumoniae.

A fecal microbiota transplantation (a mixture of fresh stools and frozen stools according to the protocol described in Example 1, originating from her husband) was carried out rectally, which resolved the Citrobacter freundii colonization, making the patient able to receive an HSC transplant 16 days later.

It should be noted that the patient did not present major infectious complications during the period of post-transplant aplasia. It should also be noted that the Klebsiella Pneumoniae which was detectable up to D60 of the transplant was no longer detected on subsequent rectal swabbing.

The patient did not develop GVH following the HSC transplant (three-month follow-up).

1. Sample of fecal microbiota for use in the prevention and/or the treatment of infectious or non-infectious complications resulting from an allogenic hematopoietic stem cell (HSC) transplant in a recipient patient, said sample of fecal microbiota being administered before the HSC transplant.

2. Sample of fecal microbiota for use according to claim 1, said sample de fecal microbiota and said hematopoietic stem cells originating from one and the same donor subject.

3. Sample of fecal microbiota for use in the prevention and/or the treatment of infectious or non-infectious complications resulting from an allogenic hematopoietic stem cell transplant (HSC) in a recipient patient, said sample of fecal microbiota and said hematopoietic stem cells originating from one and the same donor subject.

4. Sample of fecal microbiota for use according to claim 3, by transplantation of the sample in the alimentary tract of the recipient patient after said HSC transplant, preferably by transplantation of the sample in the alimentary tract of the recipient patient from 1 week to 2 years after said HSC transplant, optionally repeatedly.

5. Sample of fecal microbiota for use according to claim 1, by transplantation of the sample in the alimentary tract of the recipient patient before said HSC transplant, by transplantation of the sample in the alimentary tract of the recipient patient from 1 week to 12 months, preferably 1 week to 8 months, preferably 1 week to 6 months, more preferably from 1 week to 4 months, preferably 1 week to 3 months, or from one to eight weeks before said HSC transplant, optionally repeatedly.

6. Sample of fecal microbiota for use according to claim 1, in the prevention and/or the treatment of an infection by Clostridium or by enterococci, in particular an infection by vancomycin-resistant enterococci (VRE).

7. Sample of fecal microbiota for use according to claim 1, in the prevention and/or the treatment of a GVH disease.

8. Sample of fecal microbiota for use according to claim 1, where the recipient patient is suffering from a cancer.

9. Sample of fecal microbiota for use in the treatment of a cancer in a recipient patient suffering from a cancer and undergoing or having undergone an allogenic hematopoietic stem cell transplant, said sample of fecal microbiota and said hematopoietic stem cells originating from one and the same donor subject.

10. Sample of fecal microbiota for use according to claim 8, by transplantation of the sample in the alimentary tract of the recipient patient before said HSC transplant, preferably from 1 week to 12 months, preferably 1 week to 8 months, preferably 1 week to 6 months, more preferably from 1 week to 4 months, preferably 1 week to 3 months, or from one week to eight weeks before said HSC transplant, optionally repeatedly.

11. Sample of fecal microbiota for use according to claim 8, by transplantation of the sample in the alimentary tract of the recipient patient after said HSC transplant, preferably by transplantation of the sample in the alimentary tract of the recipient patient from 1 week to 2 years after said HSC transplant, optionally repeatedly.

12. Sample of fecal microbiota for use according to claim 1, where said recipient patient is suffering from a malignant hemopathy.

13. Sample of fecal microbiota for use according to claim 1, said sample being able to be obtained by thawing from i) mixing a stool sample from the donor subject with a saline aqueous solution comprising a cryoprotectant agent and/or a bulking agent, optionally followed by ii) filtration, before iii) freezing for storage, the steps of mixing and freezing preferably being carried out under anaerobic conditions.

14. Sample of fecal microbiota for use according to claim 1, by administration via the rectal route.
